(19) United States
(12) Patent Application Publication
(10) Pub. No.: US 2008/0286348 A1
(43) Pub. Date: Nov. 20, 2008

(54) Secured transdermal delivery system

(76) Inventor: John A. Todhunter, Fairfax Station, VA (US)

Correspondence Address: General Counsel, P.C., 6862 Elm Street, SUITE 800, McLean, VA, 22101, US

(21) Appl. No.: 11/803,855

(22) Filed: May 16, 2007

(51) Int. Cl. A61K 9/70

(52) U.S. Cl. 424/449

(57) ABSTRACT

A secured transdermal delivery system, applicable to both matrix and reservoir patches, for preventing misuse of medications embedded within and affixed to transdermal patches. The secured transdermal delivery system includes a backing supporting a pharmaceutical composition and one or more security agents adapted to modify the pharmacophore of the pharmaceutical composition. The security agent is separated from the pharmaceutical composition by one or more dissolving security layers which define one or more security reservoirs within the patch.

### FIELD OF THE INVENTION

The present invention relates to the field of transdermal delivery patches and more specifically to the field of patch-related pharmaceutical safety equipment.

### BACKGROUND

Alexandre Dumas in the The Count of Monte Cristo reminded the world that “in medicine use is made of the most violent poisons, which become, according as they are employed, most salutary remedies.” The very same compositions, which in specific doses heal, damage the human body when taken under non-medicinal conditions. Unfortunately, compositions intended as medicinal frequently are used in ways unintended by the prescribing physician. It is often the case that drugs offer a euphoria in addition to their medicinal qualities, which lures patients with more ingenuity than foresight into altering their medication for purposes of entertainment or relaxation. Taking medicine for purposes other than its inherent healing properties is a sure sign of medicinal abuse, and medicinal abuse is becoming more common.

The healthcare provider is faced with multiple avenues for injecting drugs into the bloodstream: pills; injection; intravenous delivery devices; and transdermal delivery systems. Among the forms of medicine delivery, pills are perhaps the most easily abused. Pills are generally administered in large quantities and are meant to be spaced over long periods of time. It is typically a mere warning label that stands between the patient and pill dosage alteration. For this reason, doctors often prefer alternate drug delivery methods for drugs that are commonly abused, for example opioids. The transdermal delivery devices, commonly known simply as “patches,” are the current preferred method for delivering addictive agents into the bloodstream.

Medicinal patches are relatively new; they were first widely introduced to the public in the 1980's. The basic components of these first generation patches included merely a piece of adherent plastic coated by a drug previously dissolved in alcohol. Rather than relying on ingestion or invasive entry, these patches would be temporarily affixed to the skin and provide a steady stream of drug delivery. Patches were a substantial improvement in the medicinal delivery art as the patches tended to be unnoticeably small (or easily hidden by clothing); offer a source of medicine that a user would not miss due to memory problems; and offer a very reliable, steady method of drug intake. As with most innovation in its infancy, early patches were plagued by problems; patches in particular had problems ranging from ineffective adherence to skin irritation. Further complicating matters, the compositions within the patches often require alteration to facilitate absorption by the skin. Skin is not naturally conducive to the intake of chemical compositions, rather its purpose as the largest organ is to prevent such action. Chemical compositions used in connection with patches had to be selected carefully such that their sizes and chemical properties fit very specific, necessary criteria. However, current patches have achieved a sophistication that has solved many of the early problems once prevalent in transdermal dosage delivery.

There exist currently many varieties of patches, many with distinct means of achieving their purposes. Structurally, patches come in two prominent varieties: a reservoir version, and a matrix version. Matrix patches include a backing bearing a combination of adhesive and drug within a polymeric matrix. Reservoir patches generally include an adherent backing; a reservoir holding the medicinal composition in either a gel or liquid form; and a membrane affixed to the reservoir to allow the medicinal composition controlled access to skin. Because reservoir patches, unlike matrix patches, initially separate the drug from the skin with the membrane; absorption enhancers such as alcohol or glycol are often used with the drugs within the reservoir. Irrespective of their construction, patches exist to provide a steady rate of drug absorption and consistent levels of medication in the body. Patches allow controlled dosages into the bloodstream of a patient, often in spite of the patient's desires.

A good example of innovation geared towards foiling a patient's desire to tamper with dosage rates is U.S. Pat. No. 6,093,419. The '419 patent purports to disclose a substance countering agent especially for an abused drug, alcohol, or a contraceptive agent. The patch contains a flexible adherent sheet or patch used to achieve the transdermal delivery of the agent through the skin of a person. The patch is limp, fragile, and non-self-supporting. The patch has a weak structure and is sufficiently limp and fragile so that the patch cannot be removed from the skin and later reapplied to the skin as a smooth planar covering. A compliance verification method is also provided for compulsory drug administration. This method includes providing a flexible adhesive patch containing a substance countering agent to be administered transdermally to the patient and maintaining the patch sufficiently limp and fragile so that the patch cannot be removed from the skin and later reapplied to the skin as a smooth planar covering. Prior to skin application, a supporting sheet or carrier is provided for supporting the patch in an outstretched, wrinkle-free condition. The patch is applied to the skin and the supporting sheet or carrier is removed, leaving the patch on the skin as a thin, membranous covering.

Some individuals cannot be trusted to properly take medication. The '419 patent is primarily geared towards those patients that will remove their patches, simply to rid themselves of recuperative medications. However, patients often misuse their patches for a purpose more sinister than chemical abstinence: chemical gluttony.

As noted earlier, patches are a very popular means of treating individuals with drug addictions. Often the compositions within the patches used to fight addiction comprise small quantities of the drug from which the patient is meant to be freed, or often the patch will include an at least slightly less addictive composition to fight the original drug addiction. Addicts lacking the willpower to accept small, controlled doses have devised many ways for concentrating large, unintended quantities from one or more patches. A popular method for extracting unintended quantities from one or more patches is simply soaking or boiling the patch in a solvent, typically water. For the less creative addict, simply swallowing the patch seems an attractive option.

U.S. Pat. No. 5,149,538 purports to disclose a misuse-resistive dosage form for the transdermal delivery of opioid that includes a combination of: one or more opioid permeable to the skin; delivery means permeable to the opioid; one or more antagonist for the opioid releasable upon ingestion or solvent immersion; and impermeable barrier means separating the opioid and the antagonist. United States Published Application No. 20040241218 relates to transdermal dosage form comprising at least one activating agent and at least one inactivating agent. The dosage form releases the inactivating agent upon disruption of the dosage form thereby preventing or hindering misuse of the active agent contained in the dosage form.

Although the '538 patent presents a cogent potential solution to discourage individuals from tampering with their opioid drug patches; the patches of the '538 patent are limited to the use of antagonists in limiting the effectiveness of misused opioid-bearing patches. U.S. Published Application No. 20040241218 is limited to chemicals that immobilize a pharmaceutical drug molecule so as to render it unavailable for systemic uptake. Although these references disclose means for combating pharmaceutical abuse through competition and uptake inactivation, respectively; there is a need for a patch that includes a security mechanism capable preventing misuse of patches through modification of the specific portion of a pharmaceutical compound responsible for initiating euphoria, i.e. the pharmacophore. A pharmacophore modification security mechanism would apply to a broad variety of drugs capable of use within patches.

### SUMMARY

The present invention is directed to a secured transdermal delivery system for preventing misuse of medications embedded within and affixed to transdermal patches. The secured transdermal delivery system includes a backing that supports a pharmaceutical composition and a security agent.

The backing of the present invention has two surfaces: a ventral surface and a dorsal surface. The ventral surface is adapted to adhere to the skin of a user and urge the pharmaceutical composition into contact with the skin. The pharmaceutical composition is disposed within a composition reservoir, formed by a recess in the backing. Opposite of the ventral surface of the backing is the dorsal surface. The dorsal surface is treated to prevent the ingress of unwanted environmental effects, e.g. moisture.

The security agent of the present invention includes one or more pharmacophore modification reagents. The pharmacophore modification reagent is a chemical adapted to directly modify the portion of the pharmaceutical composition responsible for initiating euphoria, i.e. the pharmacophore. Depending on the particular inactivation agent, the chemical modification of the present invention could result from destroying, adding to, or subtracting from the pharmacophore—rather than operating on the pharmaceutical composition's target molecule or some portion of the pharmaceutical composition distinct from the pharmacophore. The security agent is sheltered from the pharmaceutical agent by a dissolving security layer that forms a security reservoir within the composition reservoir. The security layer is adapted to degrade upon contact with solvents, particularly water, commonly used by addicts to extract chemicals from patches, which will cause the contents of the security reservoir(s) to intermingle with the pharmaceutical composition.

The pharmaceutical composition of the present invention is disposed within a pharmaceutical reservoir of the backing composition reservoir. The pharmaceutical reservoir can be a distinct entity or a simply space within the composition reservoir unoccupied by the security reservoir. Matrix patch embodiments of the present invention may further include a chemically modified starch matrix to suspend the pharmaceutical composition. Pharmaceutical compound delivery from a transdermal patch is well known to be a function of the nature of the pharmaceutical compound, the dermal penetration adjuvants used, the drug concentration within the delivery matrix (or reservoir), the surface area of the patch in contact with the skin, and the characteristics of the skin contacting the patch.

In the previously described matrix patch embodiment of the secured transdermal delivery system, the backing forms a composition reservoir having an opening facing the same direction as the ventral surface. In a reservoir patch embodiment of the secured transdermal delivery system, a membrane seals the opening of the composition reservoir. The membrane of the reservoir patch embodiment is adapted to allow passage of the pharmaceutical composition within the pharmaceutical reservoir through the membrane to the skin of a user.

Embodiments of the secured transdermal delivery system may further include an enhanced security configuration that may include multiple pharmacophore modification reagents simultaneously disposed within the composition reservoir. In such embodiments, the security agents are preferably separated into distinct security reservoirs, each capable of release upon the system's contact with a solvent.

Therefore, it is an aspect of the present invention to provide a patch with a security mechanism capable preventing patch misuse.

It is a further aspect of the present invention to provide a patch with a security mechanism capable preventing patch misuse that applies to a wide variety of drugs capable of use within patches.

These aspects of the invention are not meant to be exclusive. Furthermore, some features may apply to certain versions of the invention, but not others. Other features, aspects, and advantages of the present invention will be readily apparent to those of ordinary skill in the art when read in conjunction with the following description, and accompanying drawings.

### BRIEF DESCRIPTION OF THE DRAWINGS

FIG. 1 is an isometric view of the secured transdermal delivery system.

FIG. 2 is a sectional view of a matrix patch embodiment of the secured transdermal delivery system.

FIG. 3 is a sectional view of a matrix patch embodiment of the of the secured transdermal delivery system.

FIG. 4 is a sectional view of a matrix patch embodiment of the of the secured transdermal delivery system.

FIG. 5 is a sectional view of a reservoir patch embodiment of the secured transdermal delivery system.

FIG. 6 is a sectional view of a reservoir patch embodiment of the secured transdermal delivery system.

FIG. 7 is a sectional view of a reservoir patch embodiment of the secured transdermal delivery system.

### DETAILED DESCRIPTION

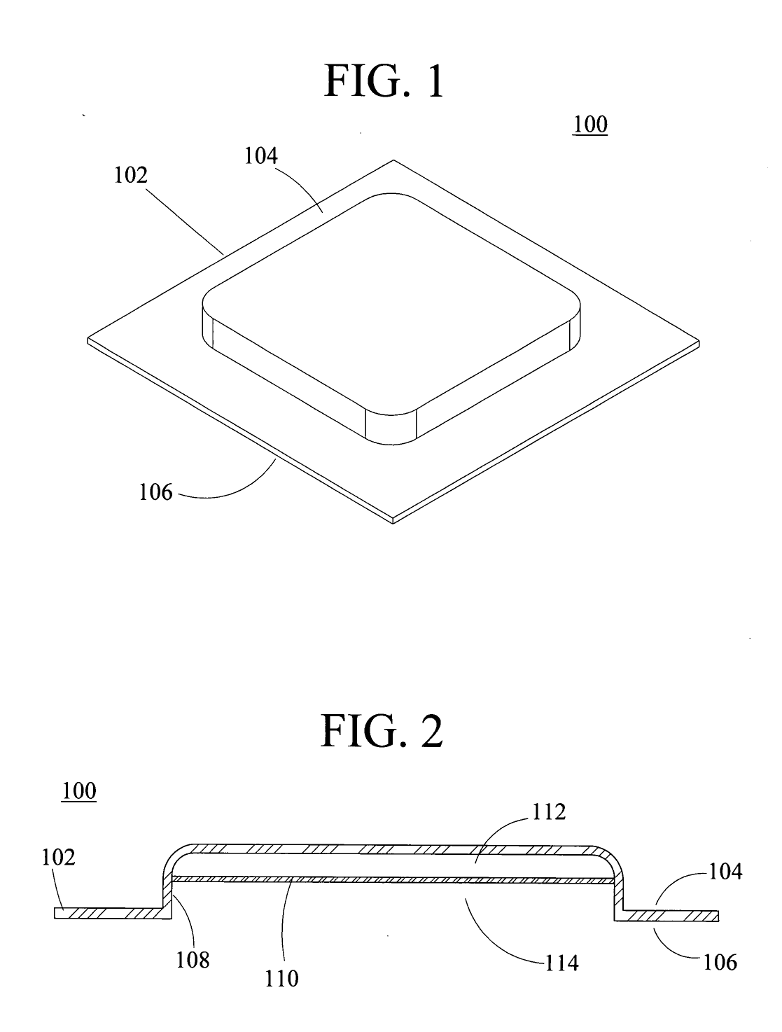

Referring first to FIG. 1, a basic embodiment of the secured transdermal delivery system 100 is shown. The secured transdermal delivery system 100 includes a backing 102 with a dorsal surface 104; and opposite the dorsal surface, a ventral surface 106. The backing has no preferred shape, common shapes (e.g. rectangular, circular, etc.) within the art of transdermal delivery patches apply to the present invention. The ventral surface 106 is the portion of the system 100 that adheres to the skin of a user and urges the pharmaceutical composition into contact with the skin. To achieve adhesion the ventral surface will include an adhesive, preferably at least about the periphery of the ventral surface. Matrix patch embodiments of the present invention, however, could include a pharmaceutical composition suspended in a composition configured to adhere to human skin. The present invention may include as its adhesive any of the adhesives, or methods of adhesion, commonly used in the skin application of transdermal patches.

The dorsal surface 104 of the backing 102 protects the contents within the backing 102 against environmental effects. These environmental effects could be any commonly experienced by the bearer of a transdermal patch, including: chemical effects such as water, grease, smoke, etc; and physical effects such as contact with fingernails, furniture, clothing, etc. There is no preferred material from which to fabricate the backing 102; materials commonly used in the manufacture of transdermal patches will similarly suit the present invention.

Turning to FIG. 2, a cross sectional view of the secured delivery system 100 is shown. As can be seen, the backing 102 forms a composition reservoir 108. The composition reservoir 108 is a recess that is formed by the curvature of the backing 102 towards the dorsal surface 104. The amount of curvature of the backing will vary with the volume of medication used with the system 100 of the present invention. In embodiments supporting relatively large volumes of medication, the backing could form a noticeable bulge; in embodiments supporting relatively small volumes of medication, the backing could be negligibly curved—or not curved at all. In cases where the backing comprises relatively no curvature, then the composition reservoir is understood to simply be the portion of backing contacting a chemical composition within the system 100. Although it is more accurate to characterize the composition reservoir of matrix embodiments of the present invention as a cavity, because the present invention includes reservoir patches, for reasons of uniformity, any portion of the backing 102 recessed to support a chemical composition will be termed “composition reservoir.” It is preferred that the backing 102 is not a planar surface, as the composition reservoir includes a dissolving security layer 110 disposed therein.

The dissolving security layer 110 of the present invention is a layer of material separating the composition reservoir 108 into at least two distinct portions: a security reservoir 112 and a pharmaceutical reservoir 114. It is preferred that the dissolving security layer 110 be constructed of a material impermeable to whatever contents are disposed within the security reservoir 112 and the pharmaceutical reservoir 114. In one embodiment of the system 100, the periphery of the dissolving security layer 110 is adhered to the backing 102 with glue that degrades upon contact with water—or other common solvents. As the glue breaks down, the dissolving security layer detaches from the backing to allow the contents of the security reservoir 112 and the pharmaceutical reservoir 114 to intermingle. In another embodiment of the system 100 of the present invention, the dissolving security layer 110 is constructed of a material that degrades upon contact with water, or other common solvent. Although the dissolving security later 110 could be constructed and configured to dissolve upon contact with water, alcohol, organic acids, ketones, glycols, or other single specific common solvent; it is preferred that the dissolving security layer be constructed to dissolve upon contact with a broad range of common solvents.

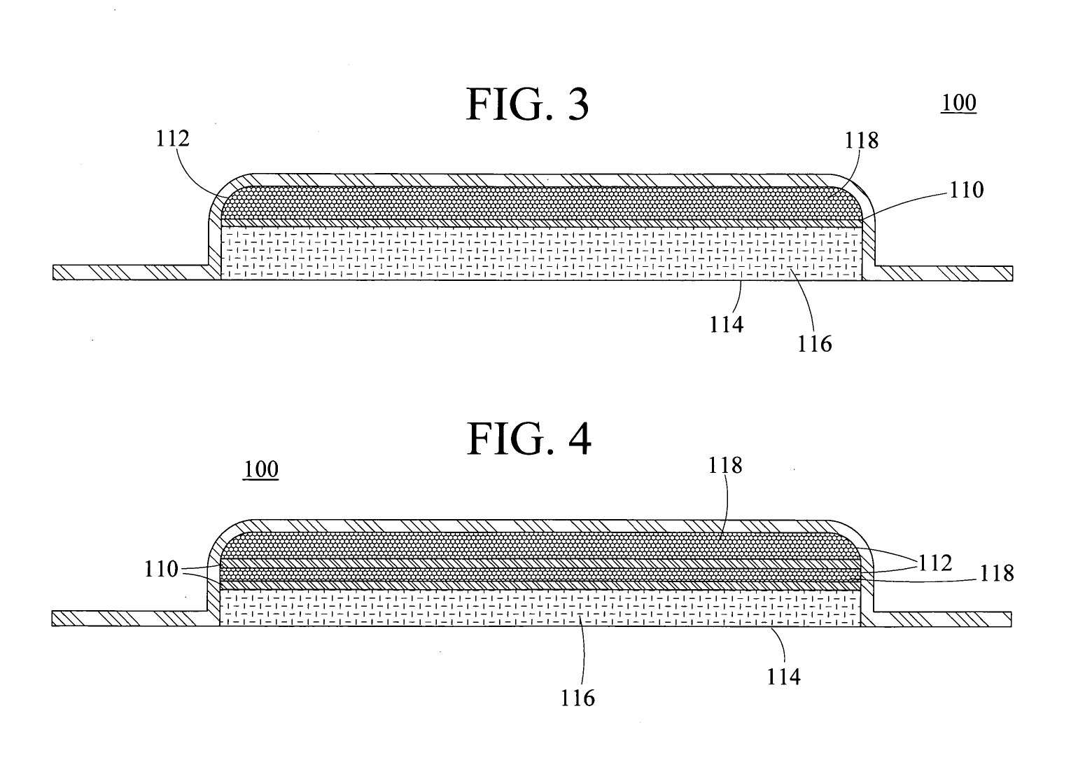

As FIG. 3 shows, the security reservoir 112 contains a security composition 118; the pharmaceutical reservoir 114 contains a pharmaceutical composition 116. There is no specific preferred pharmaceutical composition for use with the present invention. Candidates for pharmaceutical compositions include any medications commonly diverted from their intended uses. Examples of such commonly diverted medications include opioids narcotics, pseudoephedrine, and ephedrine. In matrix embodiments, such as that illustrated in FIG. 3, it is preferred that the pharmaceutical composition be contained within a delivery matrix, e.g. a silicone elastomer adhesive or other materials commonly used in transdermal drug delivery devices.

In matrix patch versions of the system 100, it is preferred that the pharmaceutical composition have a variable density. The variable density is configured to transmit an ideal dosage to a patient. The preferred means for delivering an ideal dosage is to include within the pharmaceutical composition at least two densities: a booster density positioned to immediately contact the skin upon application of the patch, and a primary density distally situated from the skin that begins delivery of a pharmaceutical composition at a point in time later than the booster density. The booster density includes a pharmaceutical composition concentration relatively greater than that of the primary density. The booster density acts to deliver a relatively quick and temporary boost of medication to provide a quick increase in the blood plasma level. The primary density then delivers the pharmaceutical composition at a rate capable of sustaining the blood plasma level.

As with generally any transdermal delivery patch, it is preferred that the ventral surface 106 be covered with a film impermeable to environmental effects. Films currently used in the transdermal delivery art will suffice with the present invention.

The security composition 118 of the system 100 of the present invention is chosen based on the pharmaceutical composition 116 within the pharmaceutical reservoir 114. Embodiments of the present invention may include one or more varieties of pharmacophore modification reagents, as FIG. 4 illustrates; both preferably segmented one from the other. The pharmacophore modification reagent of the present invention is adapted to modify the portion of the pharmaceutical composition responsible for initiating euphoria. As previously mentioned, the specific pharmacophore modification reagent will depend upon the pharmaceutical composition of the system. However, it is preferred that the pharmacophore modification reagent used is capable of nullifying the efficacy of a broad class of pharmaceutical compounds through pharmacophore modification. Examples of such broadly applicable pharmacophore modification reagents include oxidizing agents, reducing agents, alkylating agents, acylating agents, electrophiclic agents, nucleophilic agents, halogenating agents, chelating agents, or chemicals adapted to create a charge transfer complex with the pharmaceutical composition. Stable combinations, either in a single reservoir or multiple reservoirs, of the above are also be acceptable.

The preferred reducing agent of the present invention includes a biphasic system with powdered zinc in combination with sodium hydroxide. Other reducing agents include sodium borohydride and magnesium ethoxide. The preferred alkylating agent is diethyl sulfate; however, it is recognized that dimethyl sulfate would achieve intended results. The preferred acylating agent includes butyl chloride; but could include acetyl chloride, propionyl chloride, and benzoyl chloride. The preferred electrophillic agent of the present invention includes zinc chloride; stannous chloride and aluminum chloride are electrophile alternatives. Nucleophilic agents include the preferred composition sodium ethoxide, sodium methoxide, sodium methyl sulfide, and sodium peracetate. Acceptable halogenating agents include calcium hypochlorite, trichloroisocyanuric acid, potassium dichloroisocyanurate, sodium dichloroisocyanurate, and the preferred composition sodium hypochlorite. The preferred chelating agent of the present invention includes sodium citrate; sodium gluconate and ethylene diamine tetracetate (EDTA) present suitable alternatives.

Other pharmacophore modification reagents include chemicals adapted to add a nitrogen atom to a carbon atom or an oxygen atom of the pharmaceutical composition. Further pharmacophore modification reagents are adapted to add a metal atom to a carbon atom, an oxygen atom, or a nitrogen atom of the pharmaceutical composition. The above lists of candidates for pharmacophore modification reagents are not by any means exhaustive; chemicals adapted to modify pharmacophore compounds are many, and any such chemical capable of fulfilling the aspects of the present invention may be used with the system 100.

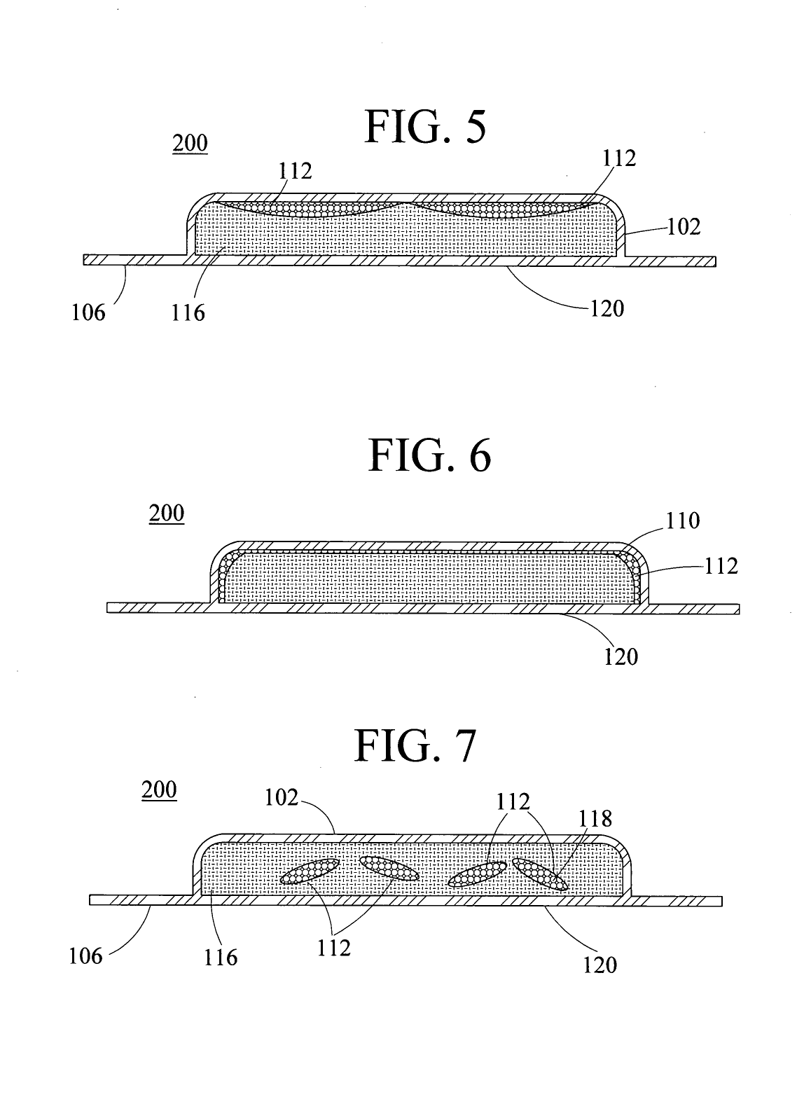

As FIGS. 5 and 6 illustrate, the security reservoirs 112 need not have any set configuration or dimensions. The security reservoir(s) 112 need only be sealed from the pharmaceutical compound until the security layer 110 of the security reservoir 112 contacts a solvent. Additional configurations and locations could include draping security reservoirs 112 affixed to the interior of the backing in one or more reservoirs, as depicted in FIG. 5; or a peripheral security reservoir 112 engulfing a substantial portion of the pharmaceutical compound 116, as depicted in FIG. 6. As FIG. 7 demonstrates, the dissolving security layers 110 could define encapsulated security reservoirs 112 within the composition reservoir 108. The capsules are fabricated of material, e.g. water-soluble polymers, capable of dissolving upon contact with water, or other common solvent.

FIG. 7 further depicts the reservoir patch system 200 of the present invention. The reservoir patch 200 includes a membrane 120 that seals the opening of the composition reservoir 108. The membrane 120 of the reservoir patch 200 is configured to allow passage of the pharmaceutical composition 116 from the pharmaceutical reservoir 108 through the membrane 120 to the skin of a user positioned proximate to the ventral surface 106 of the patch. The membrane 120 of the present invention could include any material or membrane used in the pharmaceutical arts to allow the passage of pharmaceutical compounds through. As the present invention is capable of use with many types of pharmaceutical compounds, the membrane is any adapted to mate the specific pharmaceutical compound within the system 200.

The secured transdermal delivery system includes a very broad range of pharmaceutical compounds. Upon choosing a pharmaceutical compound by which to practice the present invention, a user then need only discern one or more security agents adapted to directly modify the pharmacophore of that specific pharmaceutical composition. Below are concrete examples of specific preferred embodiments of the present invention, included within this specification merely to assist in describing the present invention.

### EXAMPLE 1

### Secured 200 mg Oxycodone Transdermal Delivery System

The backing of the secured 200 mg oxycodone transdermal delivery system includes an approximately 50 cm² ventral surface area. The pharmaceutical compound is 200 mg of oxycodone having a drug load of from 20 mg/cm³ (2%) to 80 mg/cm³ (8%) disposed within the pharmaceutical reservoir. The oxycodone is suspended within a medical grade silicone elastomer adhesive, e.g. SILASTIC ELASTOMER-382, formed into a flexible disk. Pharmacokinetic modeling suggests that in this range, a therapeutic plasma level of oxycodone can be maintained for a period between 3 to 7 days.

The secured 200 mg oxycodone transdermal delivery system includes at least a single security reservoir with a peracid as the pharmacophore modification reagent. Peracids, which promote rapid oxidation, are used in a concentration of 10:1 relative to the oxycodone. For the 200 mg portion of oxycodone, the approximately 6.4 mMole of peracids would include approximately 400 mg of peracid salt.

Any of the security reservoirs of the secured 200 mg oxycodone transdermal delivery system utilizes a dissolving security layer having a water soluble adhesive binding it to the system backing.

### EXAMPLE 2

### Secured Pseudoephedrine Transdermal Delivery System

The backing of the secured pseudoephedrine transdermal delivery system includes an approximately 50 cm² ventral surface area. The pharmaceutical compound includes between 10 to 500 mg pseudoephedrine, a concentration to effectively deliver an average plama leveal of pseudoephedrine in the range from 150 to 250 ng/mL. This is a range which covers the reported plasma concentrations in various clinical pharmacokinetic studies of pseudoephedrine and is reported to be a range over which the decongestant effect of pseudoephedrine is manifested. Although the transdermal absorption coefficients for pseudoephedrine are not yet determined, the chemical structure suggests that for pseudoephedrine base they will be similar—within a factor of 5-fold less or greater than those for oxycodone base—and the example device will contain an amount to deliver pseudoephedrine over a 3 to 5 day period.

The security layers, reservoirs, and their contents are similar to those of the 200 mg oxycodone transdermal delivery system detailed above.

Although the system of the present invention has been described in considerable detail with reference to certain preferred versions thereof, other versions would be readily apparent to those of ordinary skill in the art. Therefore, the spirit and scope of the appended claims should not be limited to the description of the preferred versions contained herein.

What is claimed is:

1. A secured transdermal delivery system comprising:
a backing defining an open composition reservoir, said backing having a ventral adherent surface and a dorsal surface substantially impervious to environmental effects;
a dissolving security layer, within said composition reservoir, dimensioned to segment said composition reservoir into a security reservoir and a pharmaceutical reservoir;
a pharmaceutical composition disposed within said pharmaceutical reservoir; and
a pharmacophore modification reagent disposed within said security reservoir,

2. The system of claim 1 further comprising a membrane, sealingly contacting said backing to form a closed composition reservoir, configured to allow selective molecular passage therethrough.

3. The system of claim 2 further comprising at least two dissolving security layers, within said composition reservoir, dimensioned to further segment said composition reservoir into multiple security reservoirs.

4. The system of claim 2 further comprising a water-soluble adhesive joining said dissolving security layer to said backing.

5. The system of claim 2 wherein said pharmacophore modification reagent is a chemical selected from a group consisting of an oxidizing agent, a reducing agent, an alkylating agent, an acylating agent, an electrophilic agent, a nucleophilic agent, a halogenating agent, a chelating agent, a chemical adapted to create a charge transfer complex with a pharmacophore of said pharmaceutical composition, and combinations thereof.

6. The system of claim 2 wherein said pharmacophore modification reagent is adapted to add a nitrogen atom to a pharmacophore target atom selected from the group consisting of a carbon atom, and an oxygen atom.

7. The system of claim 2 wherein said pharmacophore modification reagent is adapted to add a metal atom to a pharmacophore target atom selected from the group consisting of a carbon atom, an oxygen atom, and a nitrogen atom.

8. The system of claim 1 wherein said pharmaceutical composition possesses a variable density.

9. The system of claim 1 further comprising at two dissolving security layers, within said composition reservoir, dimensioned to further segment said composition reservoir into multiple security reservoirs.

10. The system of claim 1 further comprising a water-soluble adhesive joining said dissolving security layer to said backing.

11. The system of claim 1 wherein said pharmacophore modification reagent is suspended in a chemically modified starch matrix.

12. The system of claim 11 wherein said pharmacophore modification reagent is a chemical selected from a group consisting of an oxidizing agent, a reducing agent, an alkylating agent, an acylating agent, an electrophilic agent, a nucleophilic agent, a halogenating agent, a chelating agent, a chemical adapted to create a charge transfer complex with said pharmaceutical composition, and combinations thereof.

13. The system of claim 11 wherein said pharmacophore modification reagent is adapted to add a nitrogen atom to a pharmacophore target atom selected from the group consisting of a carbon atom, and an oxygen atom.

14. The system of claim 11 wherein said pharmacophore modification reagent is adapted to add a metal atom to a pharmacophore target atom selected from the group consisting of a carbon atom, a nitrogen atom, and an oxygen atom.

15. A method for preventing transdermal patch chemical diversion comprising the steps of:
embedding within a patch a pharmaceutical composition having a pharmacophore in combination with a pharmacophore modification reagent sealed from said pharmaceutical composition; and
intermingling a quantity of said pharmacophore modification reagent suitable to directly modify said pharmacophore of said pharmaceutical composition via a dissolving security layer adapted to diminish upon contact with a solvent.

16. A secured transdermal delivery system comprising:
a backing defining a pharmaceutical reservoir;
a pharmaceutical composition disposed within said pharmaceutical reservoir; and
a pharmacophore modification reagent sealingly disposed within said pharmaceutical reservoir.

17. The system of claim 16 further comprising a membrane, sealingly contacting said backing to form a closed pharmaceutical reservoir, configured to allow selective molecular passage therethrough.

18. The system of claim 16 wherein said pharmacophore modification reagent is a chemical selected from a group consisting of an oxidizing agent, a reducing agent, an alkylating agent, an acylating agent, an electrophilic agent, a nucleophilic agent, a halogenating agent, a chelating agent, a chemical adapted to create a charge transfer complex with a pharmacophore of said pharmaceutical composition, and combinations thereof.

19. The system of claim 16 wherein said pharmacophore modification reagent is adapted to add a nitrogen atom to a pharmacophore target atom selected from the group consisting of a carbon atom, and an oxygen atom.

20. The system of claim 16 wherein said pharmacophore modification reagent is adapted to add a metal atom to a pharmacophore target atom selected from the group consisting of a carbon atom, a nitrogen atom, and an oxygen atom.
